(19) United States
(12) Patent Application Publication
(10) Pub. No.: US 2002/0058040 A1
(43) Pub. Date: May 16, 2002

(54) Stable immunogenic composition for frozen storage

(76) Inventors: Stephen Grimes, Davis, CA (US); Peter Blackburn, New York, NY (US)

Correspondence Address: WHITE & CASE LLP, PATENT DEPARTMENT, 1155 AVENUE OF THE AMERICAS, NEW YORK, NY, 10036, US

(21) Appl. No.: 09/747,825

(22) Filed: Dec. 22, 2000

Related U.S. Application Data
(60) Provisional application No. 60/173,022, filed on Dec. 23, 1999.

(51) Int. Cl. A61K 39/00; A61K 39/02

(52) U.S. Cl. 424/185.1; 424/236.1

(57) ABSTRACT

An injectable vaccine composition comprising an immunogenic conjugate in an emulsion containing advantageous oily vehicles is disclosed as suitable for frozen storage; moreover, a water-in-oil emulsion composition is found to enhance immunogenicity after storage at about −18° C.

This application claims priority from the provisional application Serial No. 60/173,022 which was filed on Dec. 23, 1999.

### FIELD OF INVENTION

The invention is directed to a stable formulated immunogenic emulsion containing a combination of an antigen and an immunogenic carrier protein. More particularly, the invention is directed to a frozen emulsion which advantageously protects the immunogen during long-term storage.

### BACKGROUND OF THE INVENTION

Immunization methodology has developed from the earlier methods of vaccination against invasive organisms or particles as an effective means for generating an immune defense to more recent approaches for regulating or controlling the physiological functions and reactions of the body. The immunogenic constructs can be administered in the form of an emulsion, also containing an oily vehicle and adjuvant for potentiation on the immune response as well as emulsifying and emulsion-stabilizing agents. The immunogenic emulsions are usually either the oil-in-water or water-in-oil variety.

Although water-in-oil emulsions have posed stability problems dependent on materials, salts, temperature and other factors, water-in-mineral oil emulsions have increasingly served as effective vehicles for vaccines. The best known emulsions of this type are known in the literature as the Freund's Adjuvants which have become effectively the emulsion standard. The Complete Freund's Adjuvant differs from the Incomplete Freund's Adjuvant in that the Complete Freund's Adjuvant comprises immune response potentiating tuberculin mycobacterium. However, since these mineral oil-based adjuvant forms are not well tolerated by the parentally immunized subject, different, more amenable, forms have been introduced especially for human use. For example, U.S. Pat. No. 4,708,753 to Forsberg discloses a water-in-oil emulsion with a minor amount of emulsifying agent, wherein the oil phase is continuous. U.S. Pat. No. 4,808,334 to Ezaki, et al. is directed to a process for compositions which are sterilized at high temperature and emulsified. U.S. Pat. No. 4,960,814 to Wan et al. discloses a process to prepare a water-in-oil emulsion or, more particularly, a water-in-hydrophobic polymer emulsion. Injectable water-in-oil vaccine emulsions of low reactogenicity containing Montanide ISA 703 with 1.8% AMS are disclosed in co-assigned U.S. Pat. Nos. 5,023,077, 5,468,494 and 5,688,506. U.S. Pat. Nos. 5,422,109 and 5,424,067 to Brancq, et al. disclose an injectable vaccine emulsion comprising emulsifying agent, wherein the oil phase is continuous. U.S. Pat. No. 4,808,334 to Ezaki, et al. is directed to a process for compositions which are sterilized at high temperature and emulsified. U.S. Pat. No. 4,960,814 to Wan et al. discloses a process to prepare a water-in-oil emulsion or, more particularly, a water-in-hydrophobic polymer emulsion. Injectable water-in-oil vaccine emulsions of low reactogenicity containing Montanide ISA 703 with 1.8% AMS are disclosed in co-assigned U.S. Pat. Nos. 5,023,077, 5,468,494 and 5,688,506. U.S. Pat. Nos. 5,422,109 and 5,424,067 to Brancq, et al. disclose an injectable vaccine emulsion comprising including, e.g., Drakeol, Markol, or any mixture of squalene and squalane. U.S. Pat. No. 5,885,590 to Hunter et al. discloses injectable compositions of water-in-oil emulsions (and water-oil-water multiple emulsions) where the oily phase of the vaccine adjuvants can include squalene mostly together with a lesser amount of squalane. Under appropriate conditions immunization compositions can be enhanced by combining them with the immunological adjuvant consisting of a saline suspension of lyzed filamentous Amycolate bacteria cells.

Emulsions are formed in several different ways, such as, e.g., by mechanical action or spontaneously. Stabilization of water-in-oil emulsions formulated with a hormone peptide immunogen should preferably be achieved without applying heat, x-ray, cross-linking agents, irritating or toxic solvents and oils, in order to be pharmaceutically acceptable. Emulsion formulations of immunogens such as, e.g., anti-peptide hormone, are effective components of vaccination success. Anti-peptide hormone vaccines are herein defined as conjugates of an immunogenic carrier protein to a peptide hormone antigen comprising a hormone-immunomimic peptide.

An important practical consideration for applications of the anti-hormone vaccine technology is the shelf-life of the water-in-oil emulsion-based immunogenic composition after its manufacture and before its end use. The present refrigerated shelf-life of such formulated emulsions is about 3-6 months at about 4° C. In view of the expense of the immunogen and need for the immunogenic composition to be available for extended periods of time of treatment, it has been found desirable to obtain long term stable storage capability. The major limiting factor of a prolonged storage of the formulated emulsion vaccine has been the elution of immunomimic peptide from the immunogenic carrier.

It has now been discovered that there are several adjuvant oily substances useful as vehicles for emulsions which have been stable when frozen stored for a considerable time.

### SUMMARY OF THE INVENTION

The present invention provides an emulsified immunogenic composition which has the advantageous capability of long-term frozen storage.

According to an embodiment of the invention, it has been discovered that certain emulsified immunogenic compositions provide long-term frozen storage stability. It has been further discovered that the frozen storage of the emulsion according to the invention may be extended for more than the usual time, such as about one half year, to about one year or more.

The frozen storage capability of the inventive emulsion composition comprises metabolizable oily substances of vehicles which are pharmaceutically acceptable. The inventive emulsion can be formulated with an oily substance or vehicles containing a mixture of squalene and squalane. More particularly, an oily substance according to the present invention for producing an immunogenic emulsion which is stable during frozen storage over a wide range of freezing temperature, is selected from Montanide ISA 25, Montanide ISA 703, Montanide ISA 719, or Montanide ISA 720.

Specifically, the emulsion compositions according to this invention are found stable at the temperatures −18°, −23° and −70° C. Furthermore, the inventive composition can provide stable storage capability for an immunogen which may comprise epitopes of non-peptide or peptide antigenic moietes.

One of the embodiments of the present invention comprises a stable water-in-oil emulsion comprising a peptide hormone or peptide fragment thereof which is conjugated to an immunogenic carrier protein. Another embodiment of the invention comprises stable oil-in-water emulsion.

The conjugate in the inventive water-in-oil emulsion may comprise a synthetic hormone-immunomimic peptide linked to an immunogenic carrier.

A use of the composition includes parenteral administration. For example, in accordance with the invention, an injectable immunogen emulsion is formulated for immunization of an animal or human against its own hormone epitopes, comprising an emulsion with an aqueous phase comprising an antigen having low or negligible immunogenicity which is conjugated to an immunogenic protein carrier and an oily vehicle comprising a metabolizable oily substance or a mixture of different suitable oily substances.

Furthermore, according to the invention, the emulsion mixture remains stable after several cycles of freezing and thawing. The inventive emulsion containing the suitable oily substances have been found to be stable after undergoing several freeze/thaw cycles.

In particular, the pharmaceutically acceptable oil vehicle comprises a mixture of metabolizable squalene and squalane, and surfactant additives, such as emulsifiers and emulsion stabilizers. Furthermore, the squalene and/or squalane mixture can comprise one or more vehicles selected from the group consisting of Montanide ISA 25, Montanide ISA 703, Montanide ISA 719, and Montanide ISA 720. According to embodiment, a surfactant emulsifier can be Mannide monooleate and a surfactant emulsion stabilizer can be polyoxy-40-hydrogenated castor oil.

An embodiment of the invention provides a stable emulsion suitable for frozen storage containing a gastrin peptide or fragment thereof conjugated to an immunogenic carrier. Another embodiment provides a stable emulsion suitable for frozen storage containing a GnRH epitope or part thereof conjugated to an immunogenic carrier.

An inventive embodiment can provide a stable emulsion suitable for frozen storage containing a gastrin 17 epitope or a gastrin 34 epitope, which is conjugated to an immunogenic carrier, such as, e.g., diphtheria toxoid, tetanus toxoid, bovine serum albumin, or keyhole limpet hemocyanin, horseshoe crab hemocyanin, ovalbumin, dextran, or immunogenic fragments thereof.

Another preferred embodiment provides a stable emulsion suitable for frozen storage containing a synthetic gonadotropin releasing hormone (GnRH) peptide or fragment thereof, which is conjugated to an immunogenic carrier, such as e.g., diphtheria toxoid, tetanus toxoid, bovine serum albumin, keyhole limpet hemocyanin, horseshoe crab hemocyanin, ovalbumin or immunogenic fragments thereof.

Moreover, the frozen emulsion of this invention would remain stable for a storage period ranging up to at least 12 months at freezing temperatures ranging from about −18° C. to about −80° C. The preferred frozen emulsions of this invention remain stable for a storage period of at least 12 months at temperatures of about −18° C., −23° C. or −70° C.

One of the embodiments of the invention comprises a stable emulsion suitable for frozen storage comprising Montanide ISA 703, Montanide ISA 719 or Montanide ISA 720, which comprises pharmaceutically acceptable components, as described below. For example, the formulated emulsion may contain Montanide ISA 703, Montanide ISA 719 or Montanide ISA 720 and a synthetic G17 peptide-spacer analogue conjugated to an immunogenic moiety.

In particular, an emulsion can contain Montanide ISA 703 and human G17(1-9)-DT conjugate. Analigunot of the emulsion may contain about 0.5 mg/ml of conjugate.

Furthermore, it has been found that the immunogenic emulsion of the invention remains active when stored for an extended period at a temperature ranging from about −18° C. to about −80° C., even after several freeze/thaw cycles in succession. For example, the emulsion globules can remain at about 97% of droplet size of less than 1 μm diameter after five freeze/thaw cycles from −18° C. Furthermore, the emulsion of this embodiment comprises an intact conjugate immunogen content of about 97.5% after five −18° C. freeze/thaw cycles or about 97.5% after five −70° C. freeze/thaw cycles.

In addition, the formulated stable emulsion globules of the embodiment have retained at least 97% of their original size during frozen storage at least for 12 months.

It has been found that the anti-gastrin immunogenic emulsion of the invention surprisingly shows an improved anti-gastrin immunogenicity after one freezing/thawing cycle at −18° C. Thus, the improved immunogenicity of the inventive emulsion will significantly increase the antibody titer as compared to the starting material.

### BRIEF DESCRIPTION OF THE DRAWINGS

FIG. 1 illustrates the results of percent purity of hG17 (9)-DT conjugate in the aqueous phase extract from the emulsion after storage at −70°, −18°, 4° and 25° C., analyzed by exclusion chromatography with a TSK-GEL G3000SW_{XL} Column;

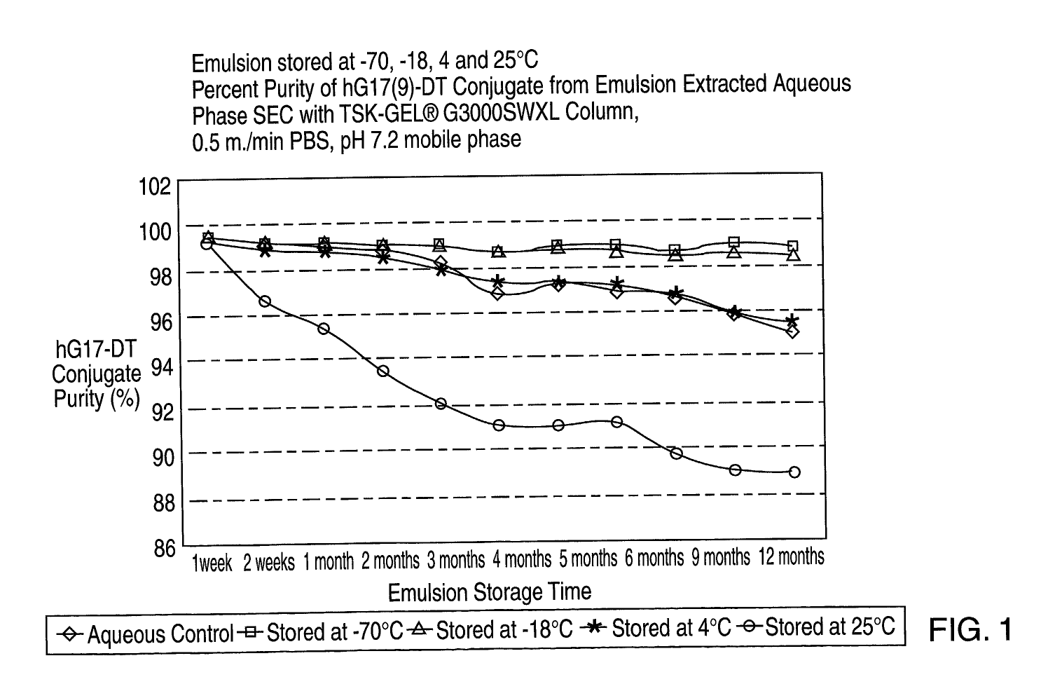

FIG. 2 illustrates the results of the material of FIG. 1, by exclusion chromatography with a TSK-GEL G2000SW column;

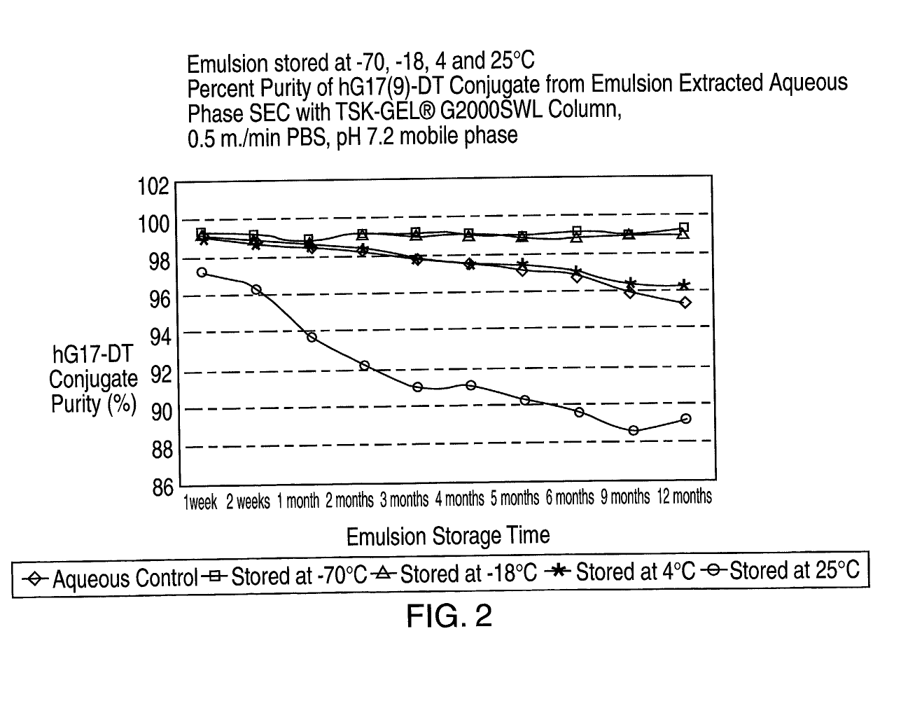

FIG. 3 illustrates percent conjugate release rate of the emulsion stored for up to 12 months at 4° C.;

FIG. 4 illustrates the conjugate release rate at 25° C.;

FIG. 5 illustrates the conjugate release rate at −70° C.;

FIG. 6 illustrates the conjugate release rate at −18° C.;

FIG. 7 illustrates the immunogenicity of emulsion after storage at 4° C. for zero, 3, 6 and 12 months;

FIG. 7 illustrates the immunogenicity of emulsion after storage at 25° C. for zero, 3, 6 and 12 months;

FIG. 9 illustrates the immunogenicity of emulsion after storage at −70° C. for zero, 3, 6 and 12 months;

FIG. 10 illustrates the immunogenicity of emulsion after storage at −18° C. for zero, 3, 6 and 12 months;

FIG. 10 illustrates the local tolerance or reactogenicity of emulsion stored at 4° C. for zero, 3, 6 and 12 months;

FIG. 12 illustrates the local tolerance or reactogenicity of emulsion stored at 25° C. for zero, 3, 6 and 12 months;

FIG. 13 illustrates the local tolerance or reactogenicity of emulsion stored at −70° C. for zero, 3, 6 and 12 months; and

FIG. 14 illustrates the local tolerance or reactogenicity of emulsion stored at −18° C. for zero, 3, 6 and 12 months.

### DETAILED DESCRIPTION OF THE INVENTION

According to this invention, immunizations against non-peptide and peptide antigens have utilized emulsions of an aqueous phase containing an immunomimic epitope conjugated to a pharmaceutically acceptable immunogenic carrier and a lipid phase containing a pharmaceutically acceptable oily substance, wherein the emulsions are formulated so as to be stable during storage with repeated freezing/thawing cycles. Pharmaceutically acceptable oily vehicles are metabolizable and understood to be well tolerated systemically by the human, as well as less irritating at the injection site of the human by showing low reactogenicity.

In accordance with the experiments described below the emulsions comprise oil-in-water, water-in-oil, and water-in-oil-in-water configurations.

Immunogenic emulsions have been disclosed in e.g., U.S. Pat. Nos. 5,422,109, 5,424,067, 5,885,590, 5,109,026, 4,708,753, 4,808,334, and 4,960,814, which are incorporated herein in their entirety by reference. More specifically, immunizations with Gastrin or GnRH immunogens in the form of injectable water-in-oil emulsions have been described in co-assigned U.S. Pat. Nos. 5,468,494, 5,023,077, 5,609,870 and 5,688,506, which are herewith incorporated in this application by reference in their entirety.

Although freezing the emulsion was originally employed as a gentle method to separate the conjugate-bearing aqueous phase from the emulsion for easier sampling and analysis, the emulsions preparations according to this invention surprisingly did not break down even when expired to several freeze-thaw cycles. This stability under the repeated freeze/thaw stress was all the more surprising because frozen storage of emulsions had not been previously considered an option. Freezing and thawing was generally held to be detrimental to the stability of emulsions, perhaps leading to disruption of conjugates and aggregation or separation of emulsion components. Moreover, when it was also found that solutions of conjugates in PBS (phosphate buffered saline) could be frozen with little loss of integrity of the conjugate of an immunogenic carrier coupled peptide, experiments were conducted to determine if it was also possible to stably store the frozen formulated emulsion. For example, the anti-gastrin formulated emulsion was tested by storage at about −70° C. (as provided by a deep freezer) or about −18° C. (as provided by a general freezer temperature). Accordingly, the emulsions of this invention have been formulated so as to keep the vaccine intact in long-term frozen storage.

In the context of the anti-hormone immunogenic embodiment of this invention, the conjugated immunogens can be synthetic peptides or fragments thereof, which may also be extended with spacer peptides, covalently attached to immunogenic protein carriers. The immunogenic carrier can be diphtheria toxoid, tetanus toxoid, a solvent extract of filamentous Amycolate or H. Pertussis, keyhole limpet hemocyanin, horseshoe crab hemocyanin, bovine serum albumin, ovalbumin, or dextran or immunogenic fragments thereof.

Dextran is a purified polysaccharide product of Leuconostoc mesenteroides strain B-512. The preferred oligosaccharide molecular weights of 64,000-76,000 are used as conjugate carrier. Other immunization enhancing additives include aluminum phosphate which serve as adsorbents for DT or TT.

The peptide or the fragment of the peptide is selected to comprise an immunomimic region of the target hormone epitope. The immunogenic conjugates are administered in the form of injectable water-in-oil or oil-in-water emulsions. Comparative tests described below have demonstrated that certain metabolizable Montanide ISA preparations (Seppic, France) has been stable during frozen storage at −23° C. or −70° C. The select group of Montanide ISA preparations include Montanide ISA 25, Montanide ISA 703, Montanide ISA 719 and Montanide ISA 720. In particular, pharmaceutically acceptable Montanide ISA 703 has been found an especially useful oily vehicle for forming a stable emulsion that is effective for immunogenic compositions. Alternatively, other metabolizable combinations of squalene/squalane and additives can be utilized which are less irritating or more gentle, and thus more amenable to the human.

A composition according to this invention comprising 0.5 mg/ml of the above described immunogenic conjugates in Montanide ISA 703 has been found to form a stable emulsion which is suitable for storage at temperatures below the freezing point. In fact, as described below, the formulated vaccine emulsion was found to remain stable when frozen for several months, up to at least about one year. Thawed-out emulsions maintained visual integrity. Storage of immunogenic emulsions at different temperatures and after one or more freeze-thaw cycles under the storage conditions described below, did not significantly affect the conjugate integrity or cause oil phase separation in the emulsion. In fact, the emulsion globules did not show any significant aggregation, did not undergo a significant shift in a size distribution, or a significant loss of desirable uniformity of confirmation by exceeding the preferred initial 1 μm size.

In addition, the immunogenicity of the emulsion was significantly increased after at least one frozen storage cycle at −₁₈° C. More specifically, immunization with the frozen sample stored at −18° C. was found to generate antibody titers which are about twice that of the emulsion which was not frozen.

Immunization emulsions suitable for frozen storage can be used with any of the anti-gastrin or anti-GnRH immunogenic conjugates, disclosed in U.S. Pat. Nos. 5,023,077 and 5,688,506, respectively.

The following examples illustrate the analysis of the inventive emulsions on the basis of certain criteria for their stability. Examples 1 and 2 employed the same preparations of emulsion. The analysis included several categories such as appearance, particle size of the emulsion globules, conjugate immunogen purity in the extracted aqueous phase, release rate of conjugate from emulsion in vitro, as well as immunogenicity and injection site tolerance in vivo.

### EXAMPLE 1

Freeze-Thaw Cycles

1. Preparation of Emulsions

The following procedure for forming an immunogenic emulsion is described in the co-assigned U.S. Pat. No. 5,023,077. In particular, the immunogenic hormone peptide conjugate (i.e., gastrin peptide immunogen conjugate) was dissolved in phosphate buffered saline at pH 7.2 (“PBS”) to produce the initial aqueous phase. The initial aqueous phase of the conjugate was dissolved in PBS at a concentration of 1.882 mg/ml. The sterile emulsion was prepared by combining the aqueous phase containing the conjugate with sterile nontoxic or non-irritant oily vehicle phase, such as, e.g., Montanide ISA 703, at a ratio of 70:30 oil to aqueous phase (w/w) to comprise the final immunogenic emulsion concentration of 0.5 mg/ml. In accordance with the present protocol, emulsions were prepared by mixing 410 ml in the Silverson 500 ml mixing head, at 8,000 rpm for 4 minutes using Montanide ISA 703 as vehicle, the conjugate was hG17(1-9)Ser9-DT.

2. Freeze-Thaw Treatments

The vials (10 per temperature tested) were stored at −70° C. (Ultra-Low Freezer), and −18° C. (standard freezer).

The samples were assessed for their appearance (Tables A and B), globule size (Table C), and conjugate concentration and purity. The vials with frozen emulsions were removed from the respective freezers and allowed to come to room temperature. The vials were mixed by moderate shaking. One vial from each temperature was kept at 4° C. for testing, while the others were used to repeat the freeze/thaw procedure at the respective temperatures. The vials were subjected to 0-5 freeze-thaw cycles.

3. Appearance

The appearance of the emulsion was noted immediately after samples were removed from either −70° or −18° C., and again after thawing to room temperature and mixed by shaking. When stored at −70° C., all components of the emulsion appeared frozen. No difference in appearance was found between the frozen and subsequently thawed emulsions and the pre-freezing emulsion control. Re-suspension by shaking was not required to maintain the original appearance.

However, not all components of the emulsion were frozen when stored at −18° C. There was a noticeable difference between the frozen and subsequently thawed emulsions in appearance from the emulsion prior to freezing. But only moderate shaking was required for uniform re-suspension of the emulsion.

Following a specific number of freeze/thaw cycles (as indicated), the samples were stored at 4° C. Under these conditions, the samples maintained a white semi-viscous appearance with no signs of settling or separation.

TABLE A
Appearance of Samples Frozen at −70° C.

| | Storage | | | |
|---|---|---|---|---|
| | Time at | | | Appearance |
| No. Of | −70° C. | Appearance | Appearance | Thawed & |
| Cycles | (hours/cycle) | Frozen | Thawed | Mixed |
| 0 | N/A | Appearance: White semi-viscous liquid no signs | | |
| | | of settling or separation | | |
| 1 | 1.08 | Solid white | White semi- | White semi- |
| | | with no signs | viscous liquid | viscous liquid |
| | | of settling | with no signs | no signs of |
| | | or separation. | of settling or | settling or |
| | | | separation.¹ | separation. |
| 2 | 1.0 | Same as | Same as | Same as |
| | | previous | previous | previous |
| | | sample | sample | sample |
| 3 | 16.5 | Same as | Same as | Same as |
| | | previous | previous | previous |
| | | sample | sample | sample |
| 4 | 1.67 | Same as | Same as | Same as |
| | | previous | previous | previous |
| | | sample | sample | sample |
| 5 | 20.42 | Same as | Same as | Same as |
| | | previous | previous | previous |
| | | sample | sample | sample |

¹During the initial part of the thaw process a very slight layer of oil was visible above the emulsion when the vial was tipped side to side. However, this oil was not visible once the sample had fully equilibrated to room temperature.

TABLE B
Appearance of Samples Frozen at −18° C.

| | Storage | | | |
|---|---|---|---|---|
| | Time at | | | Appearance |
| No. Of | −18° C. | Appearance | Appearance | Thawed & |
| Cycles | (hours/cycle) | Frozen | Thawed | Mixed |
| 0 | N/A | Appearance: White semi-viscous liquid no signs | | |
| | | of settling or separation | | |
| 1 | 22.17 | Oil layer above | Cloudy oil | White semi- |
| | | white unevenly | layer above | viscous liquid |
| | | settled | settled | with no signs |
| | | emulsion layer. | emulsion | of settling or |
| | | | containing | separation. |
| | | | dispersed | |
| | | | pockets of oil.² | |
| 2 | 24.17 | Same as | Same as | Same as |
| | | previous | previous | previous |
| | | sample | sample | sample |
| 3 | 18.83 | Same as | Same as | Same as |
| | | previous | previous | previous |
| | | sample | sample | sample |
| 4 | 70.17 | Same as | Same as | Same as |
| | | previous | previous | previous |
| | | sample | sample | sample |
| 5 | 22.50 | Same as | Same as | Same as |
| | | previous | previous | previous |
| | | sample | sample | sample |

²White emulsion with small pockets of oil unevenly distributed throughout. Oil layer comprised approximately 10-20% of total volume of liquid in vial.

4. Globular Size Distribution (Table C)

Globule size determination was performed on all samples from both freezing temperatures and the cold storage non-frozen control (4° C.). There was no change in globule size distribution after one freeze/thaw cycle, although, there was a slight increase in the percentage of globule size greater than 1 μm, ranging up to 2.5% after 5 freeze/thaw cycles.

TABLE C
Globule Size Distribution Results

| Sample | Percent ≧ 1 μm |
|---|---|
| Control Emulsion | 0.40% |
| −18° C. one F/T cycle | 0.45% |
| −18° C. two F/T cycles | 1.85% |
| −18° C. three F/T cycles | 0.89% |
| −18° C. four F/T cycles | 2.45% |
| −18° C. five F/T cycles | 2.50% |
| −70° C. one F/T cycle | 0.35% |
| −70° C. two F/T cycles | 2.17% |
| −70° C. three F/T cycles | 2.14% |
| −70° C. four F/T cycles | 2.16% |
| −70° C. five F/T cycles | 2.5% |

5. IPLC Analysis

To analyze the conjugate in the emulsions by BPLC, the conjugate-bearing aqueous phase was first extracted from the emulsion by treatment of an aliquot of emulsion with an equal volume of isobutanol. Following centrifugation (4,000×g for 10 min.) to separate the aqueous and oil phases, the aqueous phase was collected and tested by UPLC. The KPLC conditions were: flow rate=0.5 ml/min.; butter=PBS, pH=7.2; run duration=35 min.; sample volume=0.010 ml; column=TSK-GEL® G3000 SWₓ₁ (10 mm×300 mm); room temperature; injection volume=sample volume. The integrated data from the analyses was used to calculate the purity (% intact) of the conjugate extracted from the emulsions.

A retained aliquot of the aqueous phase (used to prepare the anti-gastrin immunogen) was used as an aqueous control for concentration determination (Stock conjugate lot no. G1297-5). Comparison of the chromatograms for samples subjected to five freeze/thaw cycles with chromatograms for the control showed that freezing had no effect upon the elution profile of conjugate in the sample. Moreover, under both storage conditions, there were no changes in conjugate concentration or purity after 5 freeze/thaw cycles, as seen in Tables 4 and 5.

TABLE D
Conjugate Concentration and Purity by HPLC analysis −70° C.

| Sample | Conj. Conc. In Emulsion | Purity (intact) |
|---|---|---|
| Control emulsion | 0.48 mg/ml | 99.0% |
| −70° C., five F/T cycles | 0.49 mg/ml | 98.9% |

TABLE E
Conjugate concentration and Purity by HPLC analysis −18° C.

| Sample | Conj. Conc. In Emulsion | Purity (intact) |
|---|---|---|
| Control emulsion | 0.473 mg/ml | 97.4% |
| −18° C., five F/T cycles | 0.476 mg/ml | 97.4% |

By the parameters tested, the only change observed was in globule size distribution, although it remained well within the specification of 60% less than 1 μm in size (observed 97.5% less than 1 μm at 5 freeze/thaw cycles). Therefore, these storage conditions are acceptable for emulsions under the test criteria of this study.

### EXAMPLE 2

Long Term Storage

A study was conducted to assess the stability of the inventive anti-gastrin immunogenic emulsion (e.g., hG17(1-9) Ser 9-DT conjugate) when stored at −70°, −18°, 4° and 25° C. for a period of 1 year. The mixture was prepared and emulsified under aseptic conditions 10 emulsion sample vials were stored at each temperature. The immunogenic concentration was 0.5 mg/ml emulsion volume.

At specified intervals, including at Time 0 (start of experiment), 1 week, 2 weeks, 1 month, 2 months, 3 months, 4 months, 5 months, 6 months, 9 months and 12 months, one sample vial was removed from each storage temperature and analyzed for appearance, emulsion globule size and conjugate purity. The conjugate release from the emulsion and the immunogenicity of the emulsion was analyzed at 0, 1, 3, 6, and 12 months. The results of this experiment regarding conjugate release and immunogenicity after storage at the four different temperatures are summarized below. Reference is taken to the protocol and data which are provided in the Tables below and in the figures.

| 1. Appearance (Table 1) | | |
|---|---|---|
| The appearance was | (1) | Remove one vial of emulsion |
| assessed by the following | | from each storage temperature. |
| protocol: | (2) | Record the appearance of the emulsion |
| | | samples. |
| | (3) | Allow samples to thaw to room temp. |
| | | for approximately one hour. |
| | (4) | Shake all emulsion samples by hand for |
| | | 2-3 minutes. |
| | (5) | Record the appearance of the emulsion |
| | | samples. |

After stabilization at each storage temperature, the appearance of the emulsion was visually assessed at each test storage temperature and compared to the initial emulsion at Time 0. The results can be summarized, as follows:

Sample of initial emulsion (Time 0): Homogeneous, white, semi-viscous liquid.

Sample at −70° C.: White homogenous solid. No change upon storage for 12 months.

Sample at −18° C.: Clear amber oil layer on top of the frozen white homogeneous solid. No further change upon storage for 12 months.

Sample at 4° C.: Homogeneous, white, semi-viscous liquid. No change upon storage for 12 months.

Sample at 25° C.: Homogeneous, white, semi-viscous liquid. After 5 months of storage, a small amount of creaming became apparent (i.e., settling of aqueous phase droplets in the oil continuous phase). After 12 months storage, the creaming had progressed slowly, with a small oil layer visible on top of the emulsion.

After the emulsion sample vials were removed from storage, allowed to thaw/stabilize to room temperature and shaken by hand, all samples regained their original appearance, as a white, semi-viscous liquid. Subsequent tests were run on the emulsion after warming the samples to room temperature and gentle shaking.

TABLE 1A
EMULSION APPEARANCE

| | Days | | | | |
|---|---|---|---|---|---|
| | from | Emulsion stored at 4° C. | | Emulsion stored at 25° C. | |
| Storage time | Mfg | Removal from storage | Thawed & shaken by hand | Removal from storage | Thawed & shaken by hand |
| 1 week | 8 | White semi-viscous emulsion | White semi-viscous emulsion | White semi-viscous emulsion | White semi-viscous emulsion |
| 2 weeks | 14 | White semi-viscous emulsion | White semi-viscous emulsion | White semi-viscous emulsion | White semi-viscous emulsion |
| 1 month | 28 | White semi-viscous emulsion | White semi-viscous emulsion | White semi-viscous emulsion | White semi-viscous emulsion |
| 2 months | 56 | White semi-viscous emulsion | White semi-viscous emulsion | White semi-viscous emulsion | White semi-viscous emulsion |
| 3 months | 84 | White semi-viscous emulsion | White semi-viscous emulsion | White semi-viscous emulsion | White semi-viscous emulsion |
| 4 months | 112 | White semi-viscous emulsion | White semi-viscous emulsion | White semi-viscous emulsion | White semi-viscous emulsion |
| 5 months | 140 | White semi-viscous emulsion | White semi-viscous emulsion | White semi-viscous emulsion | White semi-viscous emulsion |
| | | (a small amount of creaming | | (a small amount of creaming | |
| | | on top) | | on top) | |
| 6 months | 169 | White semi-viscous emulsion | White semi-viscous emulsion | White semi-viscous emulsion | White semi-viscous emulsion |
| | | (a small amount of creaming | | (a small amount of creaming | |
| | | on top) | | on top) | |
| 9 months | 257 | White semi-viscous emulsion | White semi-viscous emulsion | White semi-viscous emulsion | White semi-viscous emulsion |
| | | | | (a small amount of creaming | |
| | | | | on top; amber color on top but | |
| | | | | without sharp line) | |
| 12 months | 336 | White semi-viscous emulsion | White semi-viscous emulsion | White semi-viscous emulsion | White semi-viscous emulsion |
| | | | | (a small amount of creaming | |
| | | | | on top; amber color on top but | |
| | | | | without sharp line) | |

TABLE 1B
EMULSION APPEARANCE

| | Days | | | | |
|---|---|---|---|---|---|
| | from | Emulsion stored at −70° C. | | Emulsion stored at −18° C. | |
| Storage time | Mfg. | Removal from storage | Thawed & shaken by hand | Removal from storage | Thawed & shaken by hand |
| 1 week | 8 | White solid emulsion | White semi-viscous emulsion | Two phases: Clear amber oil | White semi-viscous emulsion |
| | | | | on top (˜⅓), white solid on | |
| | | | | bottom (˜⅔) | |
| 2 weeks | 14 | White solid emulsion | White semi-viscous emulsion | Two phases: Clear amber oil | White semi-viscous emulsion |
| | | | | on top (<½), white solid on | |
| | | | | bottom (>½) | |
| 1 month | 28 | White solid emulsion | White semi-viscous emulsion | Two phases: Clear amber oil | White semi-viscous emulsion |
| | | | | on top (˜½), white solid on | |
| | | | | bottom (˜½) | |
| 2 months | 56 | White solid emulsion | White semi-viscous emulsion | Two phases: Clear amber oil | White semi-viscous emulsion |
| | | | | on top (˜½), white solid on | |
| | | | | bottom (˜½) | |
| 3 months | 84 | White solid emulsion | White semi-viscous emulsion | Two phases: Clear amber oil | White semi-viscous emulsion |
| | | | | on top (˜½), white solid on | |
| | | | | bottom (˜½) | |
| 4 months | 112 | White solid emulsion | White semi-viscous emulsion | Two phases: Clear amber oil | White semi-viscous emulsion |
| | | | | on top (>½), white solid on | |
| | | | | bottom (<½) | |
| 5 months | 140 | White solid emulsion | White semi-viscous emulsion | Two phases: Clear amber oil | White semi-viscous emulsion |
| | | | | on top (>½), white solid on | |
| | | | | bottom (<½) | |
| 6 months | 169 | White solid emulsion | White semi-viscous emulsion | Two phases: Clear amber oil | White semi-viscous emulsion |
| | | | | on top (>½), white solid on | |
| | | | | bottom (<½) | |
| 9 months | 257 | White solid emulsion | White semi-viscous emulsion | Two phases: Clear amber oil | White semi-viscous emulsion |
| | | | | on top (>½), white solid on | |
| | | | | bottom (<½) | |
| 12 months | 336 | White solid emulsion | White semi-viscous emulsion | Two phases: Clear amber oil | White semi-viscous emulsion |
| | | | | on top (˜½), white solid on | |
| | | | | bottom (˜½) | |

2. Emulsion Globule Size (Table 2)

It was found that the test emulsions are stable upon storage at cold temperatures. However, it was necessary to resuspend the aqueous phase droplets by shaking (after equilibration at room temperature) prior to use. The proportion of aqueous phase droplets with a diameter≧1 μm was determined by microscopy. There was no significant change in the globule size distribution over the 12 month period for the emulsion when stored at −70° C., −18° C. and 4° C. (see Table 2). But the emulsion stored at 25° C. underwent a significant shift towards larger globules resulting in an increased proportion of droplets with a diameter≧1 μm, from 1.1% at time 0 to 28.1% after 12 months storage. Thus the results showed that the aqueous phase droplets were stable at −70° C., −18° C. and 4° C., but much less stable at 25° C.

TABLE 2
Emulsion Globule Size

| Emulsion | Emulsion | % |
|---|---|---|
| Storage | Globule size | Total |
| Time | (diameter) | Globules |
| 0 | <1 μm | 98.9 |
| | ≧1 μm | 1.1 |

| Emulsion | Emulsion | Emulsion Storage Temperatures | | | |
|---|---|---|---|---|---|
| Storage | Globule size | −70° C. | −18° C. | 4° C. | 25° C. |
| Time | (diameter) | % total | % total | % total | % total |
| 1 week | <1 μm | 98.9 | 99.2 | 99.5 | 99.7 |
| | ≧1 μm | 1.1 | 0.8 | 0.5 | 0.3 |
| 2 weeks | <1 μm | 99.7 | 99.8 | 99.9 | 99.5 |
| | ≧1 μm | 0.3 | 0.2 | 0.1 | 0.5 |
| 1 month | <1 μm | 98.5 | 99.7 | 99.4 | 99.2 |
| | ≧1 μm | 1.5 | 0.3 | 0.6 | 0.8 |
| 2 months | <1 μm | 95.4 | 97.9 | 94.1 | 99.2 |
| | ≧1 μm | 4.6 | 2.1 | 5.9 | 0.8 |
| 3 months | <1 μm | 99.6 | 99.6 | 99.2 | 91.2 |
| | ≧1 μm | 0.4 | 0.4 | 0.8 | 8.8 |
| 4 months | <1 μm | 99.5 | 98.3 | 98.9 | 64.0 |
| | ≧1 μm | 0.5 | 1.7 | 1.1 | 36.0 |
| 5 months | <1 μm | 99.6 | 98.6 | 98.2 | 80.8 |
| | ≧1 μm | 0.3 | 1.4 | 1.8 | 19.2 |
| 6 months | <1 μm | 99.7 | 98.8 | 99.9 | 90.6 |
| | ≧1 μm | 0.3 | 1.2 | 0.1 | 94 |
| 9 months | <1 μm | 97.0 | 98.5 | 99.4 | 72.0 |
| | ≧1 μm | 3.0 | 1.5 | 0.6 | 28.0 |
| 12 months | <1 μm | 99.5 | 99.5 | 98.1 | 71.9 |
| | ≧1 μm | 0.5 | 0.5 | 1.9 | 28.1 |

3. Conjugate Purity (Tables 3 and 4)

The aqueous phase was extracted from the formulated emulsion for the purity analysis of the conjugate, as described in Example 1, Item 3. Purity was determined as the proportion of intact conjugate present in each test sample by measuring the extracted aqueous phase by size exclusion chromatography in an TPLC system. Two columns, with differing separatory characteristics, were used in the analysis (the TSK-GEL® G2000SW and TSK-GEL®D G3000SWXL columns). Almost identical results were obtained with each column as tabulated below. A retained sample of the aqueous phase, stored at 4° C., served as a control.

Summary

Initial (Time 0): Conjugate purity of 99.3%.

Sample at −70° C.: No significant change after 12 months (from 99.3% to 98.9%). (Change=−0.4%)

Sample at −18° C.: Minimal change, from 99.3% to 98.5% after 12 months. (Change=−0.8%)

Sample at 4° C.: Change from 99.3% to 95.5% after 12 months. (Change =−3.8%.)

Sample at 25° C.: Significant change, from 99.3% to 89.0% after 12 months. (Change=−10.3%.)

Conclusion: The conjugate purity was most stable at −70° C. and −18° C., less stable at 4° C. and much less stable at 25° C. The conjugate purity at the various time points assessed by TPLC chromatography is summarized in Tables 3 and 4. Data were obtained from a G3000SWXL or G2000SW column, respectively.

TABLE 3
(G3000 SWXL)

| Emulsion | Conjugate Purity (%) | |
|---|---|---|
| Storage | Aqueous | Extracted |
| Time | Control | Aq. Phase |
| Time 0 | 99.5 | 99.3 |

| Emulsion | Percent Conjugate Purity | | | | |
|---|---|---|---|---|---|
| Storage | Aqueous | Emulsion Storage Temperatures | | | |
| Time | Control | −70° C. | −18° C. | 4° C. | 25° C. |
| 1 week | 99.2 | 99.1 | 99.1 | 98.9 | 96.7 |
| 2 weeks | 99.0 | 99.2 | 99.1 | 98.8 | 95.4 |
| 1 month | 98.8 | 99.0 | 99.0 | 98.5 | 93.5 |
| 2 months | 98.3 | 99.1 | 99.0 | 98.0 | 92.1 |
| 3 months | 96.9 | 98.7 | 98.7 | 97.4 | 91.1 |
| 4 months | 97.3 | 99.0 | 98.9 | 97.4 | 91.1 |
| 5 months | 97.2 | 99.0 | 98.8 | 97.0 | 91.2 |
| 6 months | 96.7 | 98.7 | 98.5 | 96.8 | 89.8 |
| 9 months | 95.9 | 99.1 | 98.7 | 96.0 | 89.1 |
| 12 months | 95.1 | 98.9 | 98.5 | 95.5 | 89.0 |

TABLE 4
(G 2000SW)

| Emulsion | Percent Conjugate Purity | | | | |
|---|---|---|---|---|---|
| Storage | Aqueous | Emulsion Storage Temperatures | | | |
| Time | Control | −70° C. | −18° C. | 4° C. | 25° C. |
| 1 week | 99.2 | 99.4 | 99.3 | 99.1 | 97.3 |
| 2 weeks | 98.9 | 99.2 | 99.2 | 98.7 | 96.3 |
| 1 month | 98.6 | 98.9 | 98.8 | 98.4 | 93.7 |
| 2 months | 98.4 | 99.2 | 99.2 | 98.2 | 92.2 |
| 3 months | 97.8 | 99.2 | 99.0 | 97.7 | 91.0 |
| 4 months | 97.5 | 99.1 | 99.0 | 97.5 | 91.0 |
| 5 months | 97.1 | 98.9 | 98.8 | 97.3 | 90.2 |
| 6 months | 96.7 | 99.1 | 98.7 | 96.9 | 89.5 |
| 9 months | 95.8 | 98.9 | 98.9 | 96.3 | 88.5 |
| 12 months | 95.2 | 99.2 | 98.8 | 96.1 | 89.1 |

4. Conjugate Release Rate from Emulsion

The rate of conjugate release from the formulated test emulsion prepared in Example 2 and was determined by stirring the emulsion in the presence of buffer and measuring the amount of hG17(1-9) Ser 9-DT released from the emulsion into the buffer at intervals of up to 1 month. Samples of approximately 0.05 ml were taken every 7 days and assessed by Radioimmunoassay (RIA).

Materials:

FTA Hemagglutination Buffer (Becton Dickenson Microbiology Systems, Cockeysville, Md.); Bovine Serum Albumin, Fraction V (“BSA”) (e.g., ICN Biochemicals, Costa Mesa, Calif.); Sodium azide, NaN₃ (M.W. 65.02) (e.g., Mallinckrodt Inc., Paris, Ky.); 12×75 mm disposable glass tubes; ¹²⁵I-labeled hG17 (NEN); Anti-hG17 monoclonal antibody mix: equal volumes of Mab # 400-1, 2, 3, 4 (1:100=40 μl Mab in 16 ml buffer); 10 ml Reacti-Vials with triangular stir bars, autoclaved; Reacti-therm heater/stirrer (Pierce); Centrifuge (e.g. Sorvall RT6000 Refrigerated Centrifuge, with H1000 rotor head); Supplemented calf serum (“SCS”), heat activated, sterile filtered (GIBCO); Polyethylene glycol PEG (M.W. 8000) (e.g., Sigma)

Reagent Solutions:

(1) 5% (W/V) NaN₃: 5.00 g NaN₃ were dissolved in 100 ml purified water; (2) 1% (W/V) BSA with 0.02% NaN₃ in FTA (“1% BSA solution”): 9.23 g FTA and 10 g of BSA were dissolved in approximately 750 ml of purified water; 4 ml 5% NaN₃ were added and the volume adjusted to 1.000 liter with water. (3) 6.5% (W/V) BSA with 0.05% NaN₃ in FTA (“6.5% BSA in FTA solution”): 1.846 g FTA, 13 g BSA were dissolved in approximately 190 ml of purified water, 2 ml of 5% NaN₃ were added and the volume was adjusted to 200 ml with purified water, and sterile filtered. (4) A solution of 25% w/v PEG+0.02 % NaN₃ (PEG MW 8,000; 250 g/L) was prepared.

Method

A. Emulsion Release Test (“ERT”)

1. 2.30 ml of sterile 6.5% BSA in FTA solution was added to sterile 10 ml Reacti-Vials, each containing a stir bar.

2. The solution was overlaid with 0.200 ml sterile Anti-gastrin immunogen emulsion and the vial contents were stirred rapidly at 37° C., n=4 vials.

3. At various intervals, stirring was stopped and the vials were centrifuged (1,500×g=2,600 rpm) for 10 minutes at room temperature to separate the emulsion from the FTA.

4. 50 μl samples of 6.5% BSA in FTA solution were obtained aseptically from each vial under the laminar flow hood, and stirring was reinitiated until the next sample time, when the sampling procedure was repeated.

B. ERT Radioimmune assay (“RIA”) p The concentration of hGI7-DT in each sample was determined by inhibition RIA as follows:

1. To 12×75 mm glass tubes is added (duplicate samples):

a. 100 μl RIA buffer (1% BSA solution). RIA buffer was also used for all sample/reagent dilutions.

b. 100 μl of stock hGI7-DT inhibitor in a dilution series of (in ng/ml): 0-35.4-50-70.7-100-141.4-200-282.8-400-565.7-800, to establish a standard curve. The 1.88 mg/ml G17-DT stock was used to dilute 1600 ng/ml (a 1:1175 dilution), followed by serial 1/√2 dilutions. For the blank (0 ng/ml) tubes, add 100 μl of buffer was used instead. Alternatively,

c. 100 μl of diluted sample buffer was used from the emulsion release samples. The dilutions were employed dependent on the concentration of the emulsion. The dilutions were adjusted with increased time, according to the rate at which conjugate was released from the Anti-gastrin immunogen into the buffer. For example, dilutions of 1:5 to 1:100 were used at first; thereafter, the dilutions are increased based upon the results of the previous sample.

d. Sample aliquots of 100 μl of ¹²⁵I-labeled hGI7 (11,500 CPM added per tube) are measured. Total counts added were determined from two 100 μl samples.

e. The 100 μl aliquot of anti-gastrin Mab was used at a predetermined dilution of about 25% binding efficacy.

2. The contents were mixed and incubated at room temperature for 2 hours.

3. 100 μl of cold (1-8° C.) SCS was added/tube and mixed.

4. 500 μl of cold (1-8° C.) 25% PEG was added to each tube and mixed until precipitated.

5. The tubes were immediately centrifuged for 30 minutes, 2700×g (3,600 rpm with the Sorvall RT6000, H1000 rotor), at 4 ° C.

6. Supernatants were aspirated and discarded.

7. The vials were counted in an automatic gamma-counter (Wallac Model: 1470 Wizard, Serial # 4700248, Aphton equipment # EQ0024).

C. Data Analysis

The standard inhibition curve was plotted (counts bound versus inhibitor added), from which the quantity of hGI7-DT in the emulsion release test FTA samples was determined. The cumulative percent of hGI7-DT release was also calculated, relative to the starting quantity, for each sample time.

$$\% Released = \frac{Total Released \times 100}{Total Conjugate Added}$$

The Total Conjugate Added is the quantity present in the anti-gastrin immunogen added to the vial. Total Released=quantity of released conjugate in the vial +quantity of released conjugate removed from the vial due to sampling

Quantity of released conjugate in the vial=

(concentration on day n)×(volume of buffer remaining in vial on day n)

Where day n was the sampling day for which the % released was determined.

Quantity of released conjugate removed from

the vial=[(conc. in buffer in first sample)×(0.05 ml)+. . . +

(conc. in buffer on day n−1)×(0.05 ml)]

Results:

Release Rate (Table 5)

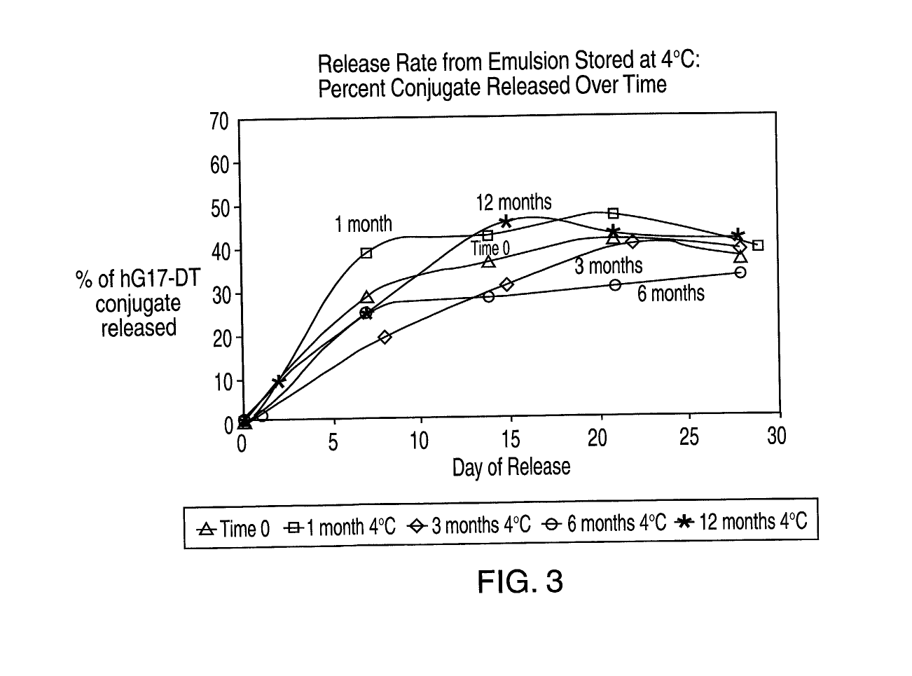

Initial control sample (Time 0): The release rate of conjugate from freshly made emulsion was determined. A maximum of 46% of conjugate was released. These data were compared to the release rate plots of emulsion for each storage temperature tested. (see FIGS. 3-6).

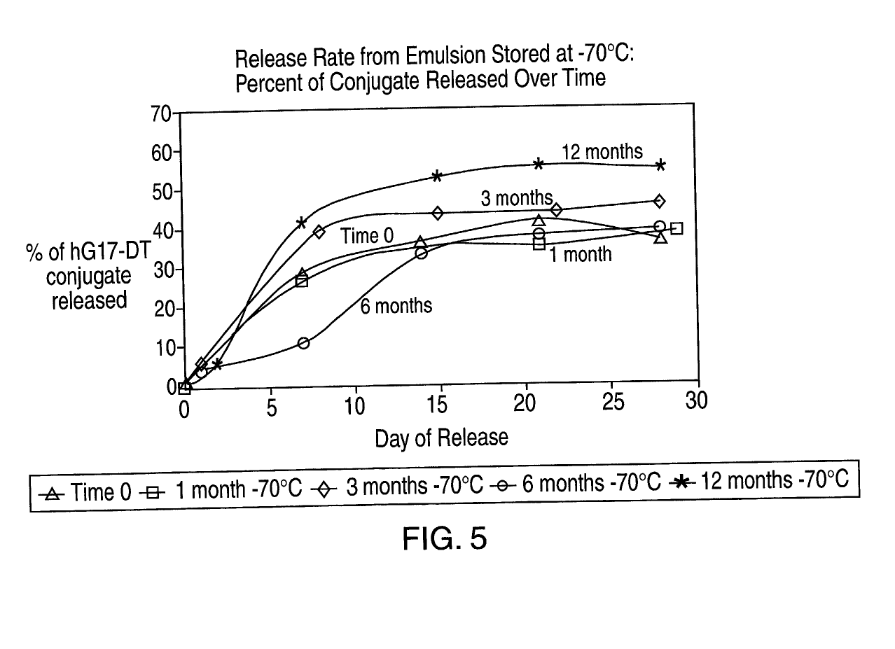

Sample at −70° C. (FIG. 5): Similar release kinetics were observed for samples stored for 0, 1, 3 and 6 months. No significant change was observed after 6 months. Samples stored for 12 months were found to release conjugate at a slightly higher rate and up to a higher total level than each of the other storage time points. The conjugate release rate and total quantity of conjugate released from emulsion stored for 12 months differed from the time emulsion release rate to a greater degree than did the emulsion stored for shorter periods of time. But in view of the differences between the initial data and those of emulsion stored for 3 and 6 months, the 12 month data do not significantly deviate from the shorter storage emulsions.

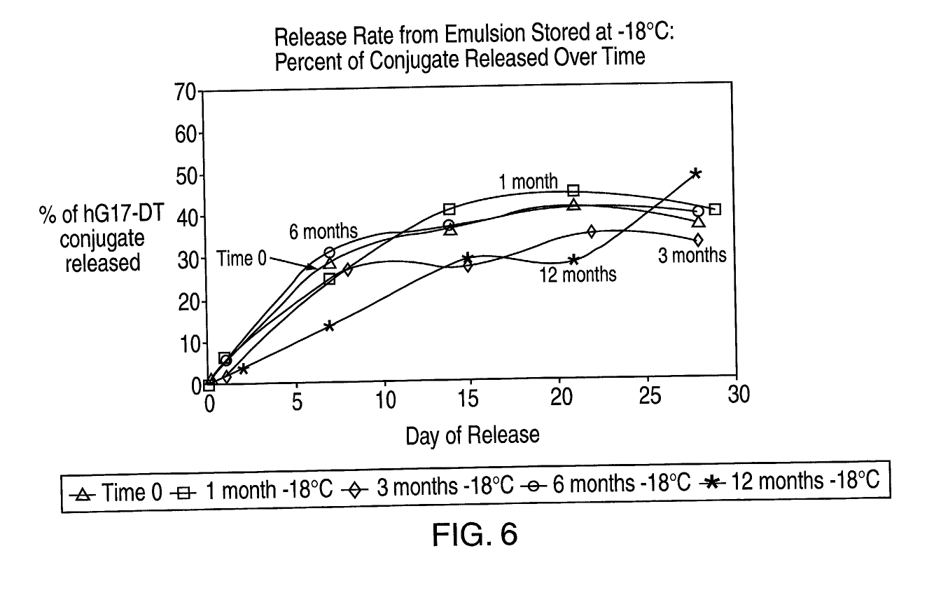

Sample at −18° C. (FIG. 6): There was no consistent pattern of conjugate release rate in an emulsion stored for shorter periods. No change over 12 months storage.

Sample at 4° C. (FIG. 3): There is no consistent change or pattern of conjugate release for emulsion stored for each time period. Thus, there is no significant change of release over the 12 month test period.

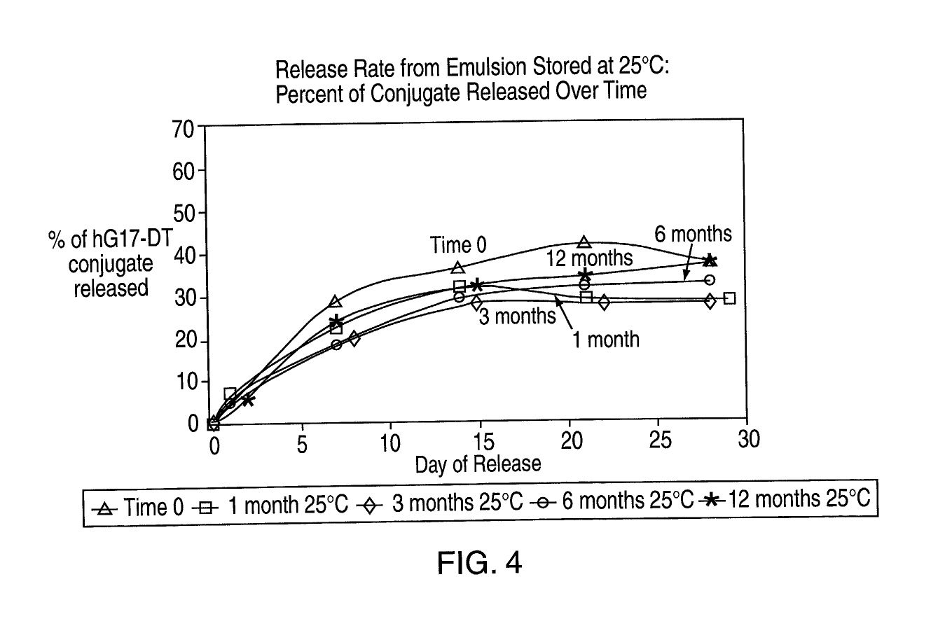

Sample at 25° C. (FIG. 4): Samples stored for 1, 3, 6 and 12 months released conjugate a somewhat slower rate, and to a lower total level than the initial time zero sample value. However, there was no discernible declining trend of release rates with increased storage time as the release curves in FIG. 4 essentially overlap. However, in this assay, storage at 25° C. altered the conjugate retaining behavior of the emulsion.

TABLE 5
EMULSION CONJUGATE RELEASE RATE - SUMMARY

| Sampling | % of hG17-DT Conjugate Released from Emulsion | | | | |
|---|---|---|---|---|---|
| Date | Time 0 | 1 month −70° C. | 3 months −70° C. | 6 months −70° C. | 12 months −70° C. |
| 0 | 0.0 | 0.0 | 0.0 | 0.0 | 0.0 |
| 0.05 | 1.2 | — | — | — | — |
| 1 | — | 5.6 | 6.3 | 4.3 | — |
| 2 | — | — | — | | 6.3 |
| 7 | 28.5 | 26.7 | — | 11.0 | 41.3 |
| 8 | — | — | 38.8 | — | — |
| 14 | 35.9 | 34.9 | — | 33.0 | — |
| 15 | — | — | 43.0 | — | 52.3 |
| 21 | 41.2 | 34.8 | — | 37.5 | 55.3 |
| 22 | — | — | 43.4 | | — |
| 28 | 36.6 | — | 45.5 | 39.2 | 54.0 |
| 29 | — | 38.4 | — | — | — |
| Sampling | % of hG17-DT Conjugate Released from Emulsion | | | | |
| Date | Time 0 | 1 month −18° C. | 3 months −18° C. | 6 months −18° C. | 12 months −18° C. |
| 0 | 0.0 | 0.0 | 0.0 | 0.0 | 0.0 |
| 0.05 | 1.2 | — | — | — | — |
| 1 | — | 6.2 | 2.2 | 5.5 | — |
| 2 | — | — | — | | 4.2 |
| 7 | 28.5 | 24.7 | — | 30.8 | 13.2 |
| 8 | — | — | 26.8 | — | — |
| 14 | 35.9 | 40.4 | — | 36.5 | — |
| 15 | — | — | 27.3 | — | 28.6 |
| 21 | 44.0 | 44.4 | — | 40.8 | 28.1 |
| 22 | — | — | 34.7 | — | — |
| 28 | 36.6 | — | 32.5 | 39.2 | 47.8 |
| 29 | — | 39.5 | — | — | — |
| Sampling | % of hG17-DT Conjugate Released from Emulsion | | | | |
| Date | Time 0 | 1 month 4° C. | 3 months 4° C. | 6 months 4° C. | 12 months 4° C. |
| 0 | 0.0 | 0.0 | 0.0 | 0.0 | 0.0 |
| 0.05 | 1.2 | — | — | — | — |
| 1 | — | 3.3 | 2.4 | 1.8 | — |
| 2 | — | — | — | — | 9.2 |
| 7 | 28.5 | 38.7 | — | 24.7 | 24.9 |
| 8 | — | — | 19.4 | — | — |
| 14 | 35.9 | 42.1 | — | 28.1 | — |
| 15 | — | — | 30.8 | — | 45.4 |
| 21 | 41.2 | 46.5 | — | 30.3 | 422 |
| 22 | — | — | 40.0 | — | — |
| 28 | 36.6 | — | 38.5 | 32.5 | 40.9 |
| 29 | — | 38.7 | — | — | — |
| Sampling | % of hG17-DT Conjugate Released from Emulsion | | | | |
| Date | Time 0 | 1 month 25° C. | 3 months 25° C. | 6 months 25° C. | 12 months 25° C. |
| 0 | 0.0 | 0.0 | 0.0 | 0.0 | 0.0 |
| 0.05 | 1.2 | — | — | — | — |
| 1 | — | 7.3 | 6.4 | 7.2 | — |
| 2 | — | | — | | 6.4 |
| 7 | 28.8 | 22.8 | — | 10.0 | 33.0 |
| 8 | — | — | 19.9 | — | — |
| 14 | 35.9 | 31.4 | — | 29.0 | — |
| 15 | — | — | 27.8 | — | 31.8 |
| 21 | 41.2 | 28.5 | | 31.2 | 33.7 |
| 22 | — | — | 27.4 | — | — |
| 28 | 38.6 | — | 27.3 | 32.2 | 36.2 |
| 29 | — | 27.7 | — | — | — |

In view of the results from the release rate tests, it was concluded that the behavior of the emulsion in the release assay was not significantly altered by storage at any of the four select temperatures. See FIGS. 3-6 and Table 5 in support of this conclusion.

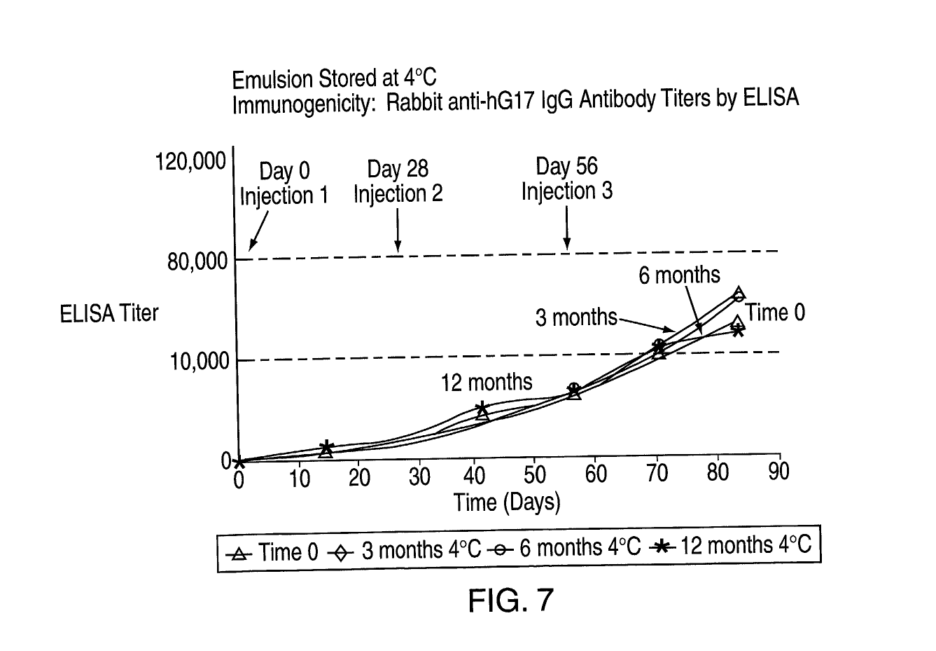

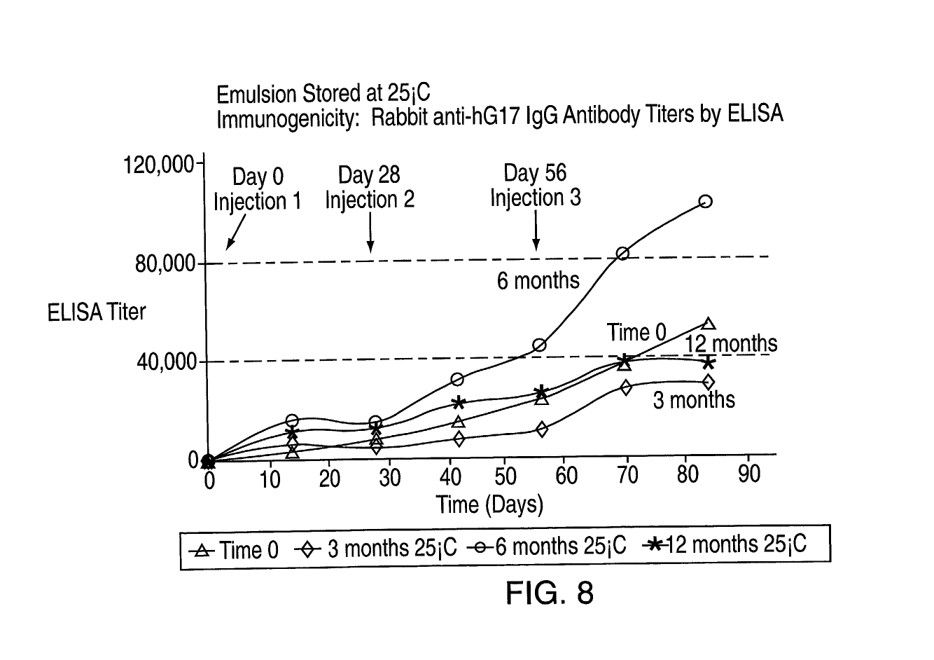

5. Immunogenicity (FIGS. 7-10)

Immunogenicity was assessed on samples stored for 0, 3, 6 and 12 months at the temperatures indicated below, in rabbits (female) by measuring serum anti-hGI7 antibody titers in a direct binding ELISA on days 0, 14, 28, 42, 56, 70 and 84 (Bleeding the animals prior to injection on injection dates). The immunogenicity data generated by freshly made emulsion (Time 0) was compared to that obtained by testing the stored material at the various temperatures (see FIGS. 7-10). The dosing schedule provided for i.m. injections of 0.25 ml (0.125 mg) emulsion sample on day 0, 28 and 56.

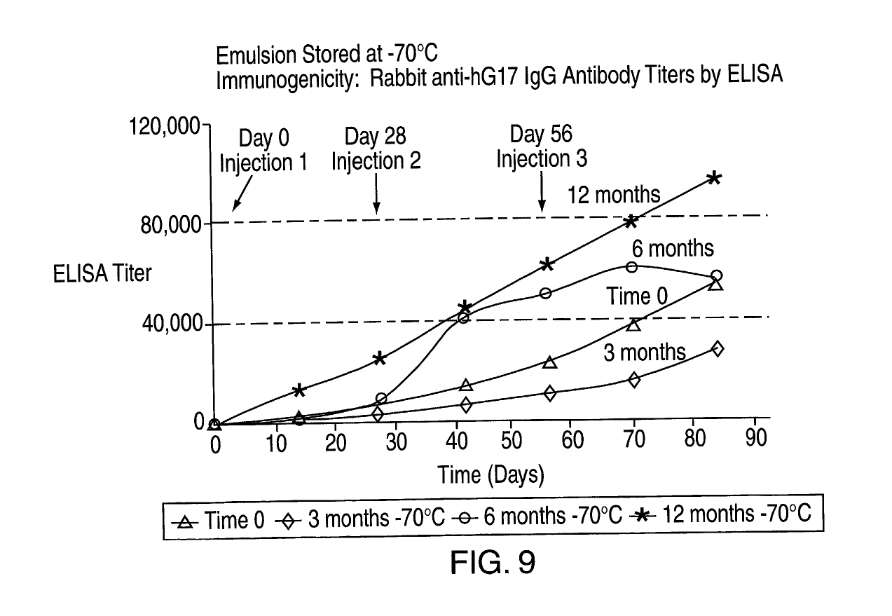

Sample at −70° C. (FIG. 9): The immunogenicity of emulsions stored at −70° C. was variable. Antibody levels for emulsion stored 3 months were lower than those at Time 0, while antibody levels for emulsion stored 6 months were slightly higher at intermediate time periods but reached the same peak value on day 84. Antibody levels at 12 months were two-fold higher than Time 0.

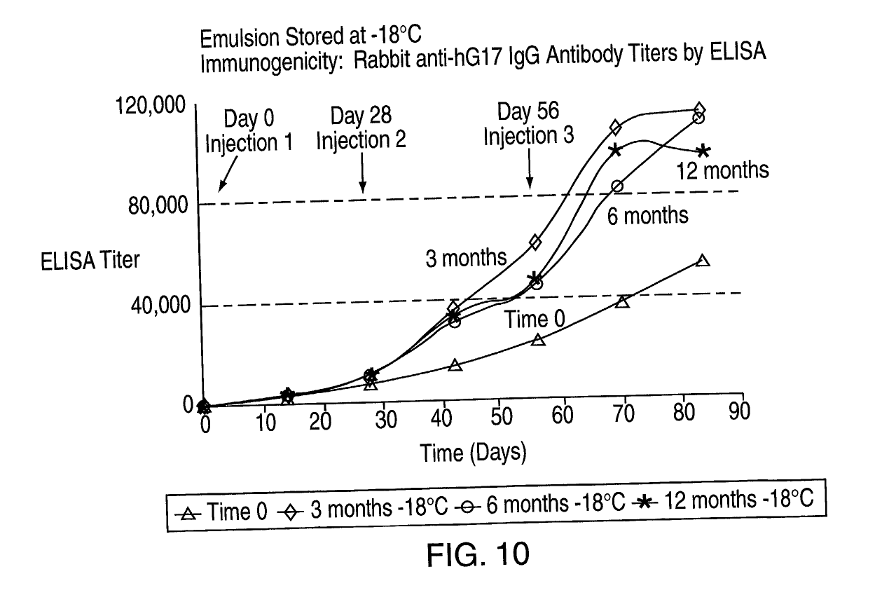

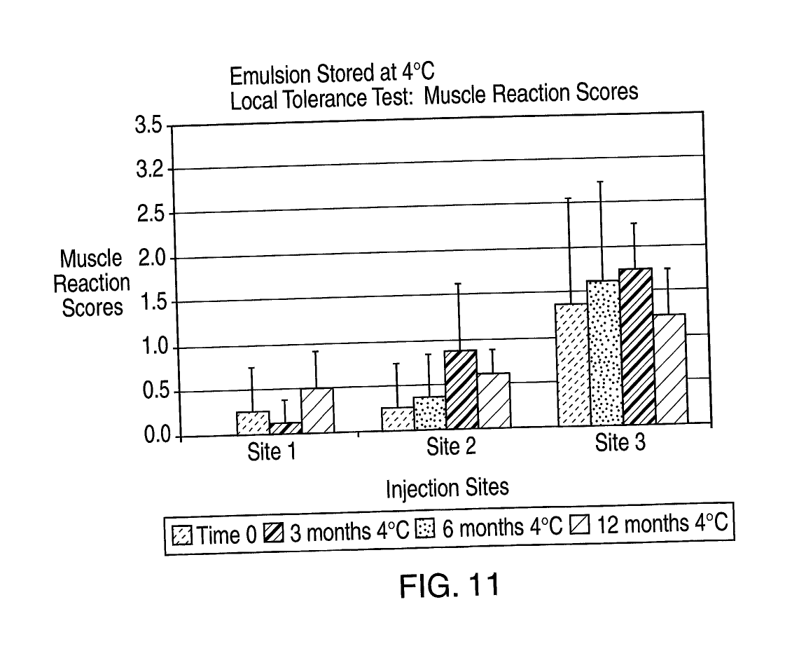

Sample at −18° C. (FIG. 10): Storage at −18° C. consistently enhanced the immunogenicity by two-fold over the starting material for emulsion held for all three incubation times. This was an unexpected finding.

Sample at 4° C. (FIG. 7): No change in immunogenicity was observed for emulsion stored at 4° C., indicating that immunogenicity was unaffected.

Sample at 25° C. (FIG. 8): Storage at 25° C. resulted in variable immunogenicity characteristics. Antibody levels at 3 months were lower than Time 0. Antibody levels at 6 months were two-fold higher than Time 0 antibody levels at 12 months were similar to Time 0 at intermediate time periods, but lower by day 84.

Conclusion: The immunogenicity response was unaffected by storage at 4° C. Storage at −18° C. increased immunogenicity. The finding that it was possible to enhance immunogenicity by a single freeze-thaw cycle (freezing at −18° C.) was unexpected. Although storage at −70° C. and 25° C. resulted in more variable responses, there was no clear trend that might be predictive for length of feasible storage time; in addition, immunogenicity was not altered from the Time 0 control.

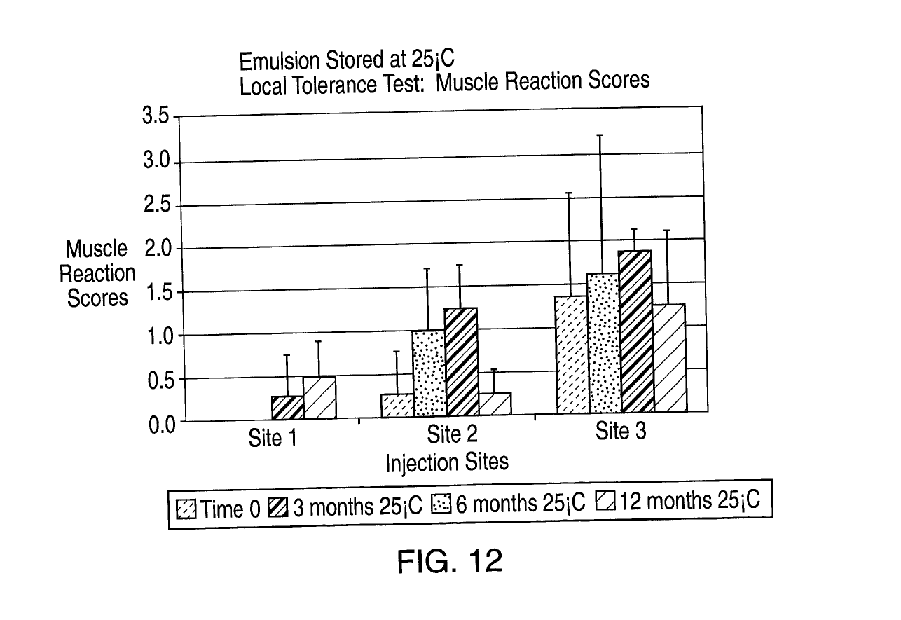

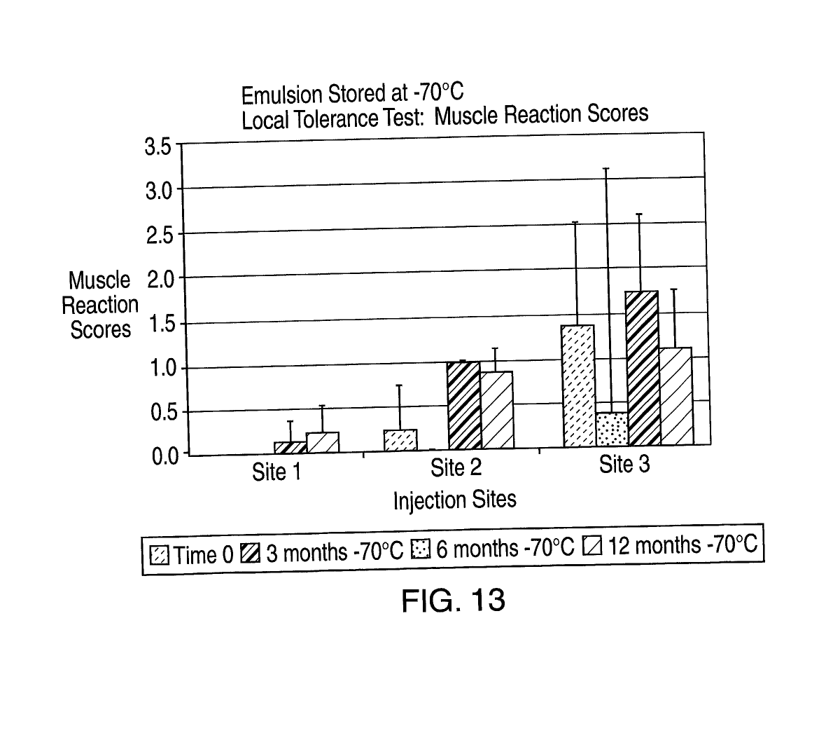

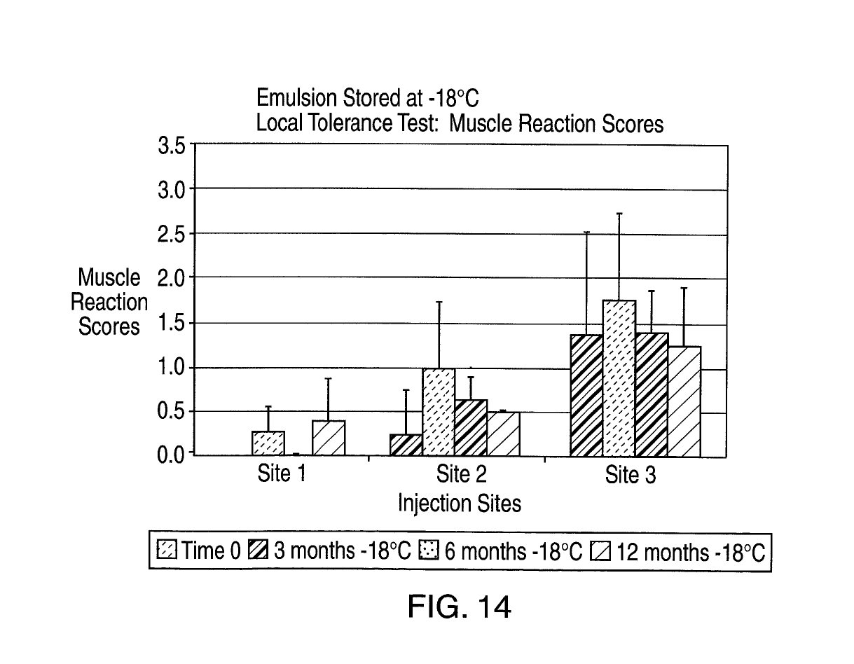

Local Tolerance. (FIGS. 11-14)

Gross injection site examinations were performed on each injection site on the euthanized subject animals on day 84 for pathology analysis. Injection site reactions were scored on a scale of 0 to 3, where 0 is normal tissue appearance and 3 is extensive in inflammation through the injected muscle.

The mean muscle reaction scores, which assess tolerance at the injection depot (reacto-genicity), increased with the injection number and correlated with the mean antibody titers. It was found that the reaction scores for emulsions held at each storage temperature were not significantly different. For example:

Sample at −70° C.: The mean injection site scores for sites 1 to 3 were 0.1, 0.6 and 1.1, respectively.

Sample at −18° C.: The mean injection site scores for sites 1 to 3 were 0.2, 0.7 and 1.5, respectively.

Sample at 4° C.: The mean injection site scores for sites 1 to 3 were 0.3, 0.6 and 1.5, respectively.

Sample at 25° C.: The mean injection site scores for sites 1 to 3 were 0.3, 0.8 and 1.6, respectively.

Conclusion: Storage temperature had no significant effect on the tissue local tolerance.

### EXAMPLE 3

Comparative experiments have been performed to investigate formulations of an immunogenic emulsion utilizing different oily vehicles to test storage stability at 4° C. and when subjected to freeze-thaw cycles at −70° C./22° C. and −23° C./22° C. Specifically, the antigastrin immunogen, G17(1-9)-DT, was mixed with different vehicles and subjected to freeze-thaw cycles at −70° C. and −23° C.

Immunogens were prepared as listed in Table 6. Accordingly, a conjugate preparation of hGI7(9)-DT (Peninsula Lab.) was mixed with an adjuvant selected from various formulations of oily substances such as different Montanide ISA preparations (Seppic, France), SB62(SmithKline Beecham, U.K.), Freund's Adjuvant, incomplete (GIBCO Lab., Grand Island, N.Y.), and Freund's Adjuvant complete (DIFCO Lab., Detroit, Mich.). The buffered oily adjuvants are also referred to as oily vehicles in the test emulsions of this disclosure.

The emulsion aqueous phase was in PBS (pH 7.2); and the SBAS3 adjuvant (the formulated SB62) was buffered in 10 mM PO₄, 150mM NaCl, pH 6.8.

Except for SBAS3, which has a 3 ml volume test emulsions were prepared at about 10 ml quantity at 0.5 mg/ml (w/w) conjugate concentration. The test emulsions were distributed in eleven vials of 0.9 ml fill volume, while the SBAS3 emulsion was distributed in 0.27 ml aliquots.

All the test emulsions were prepared by weight and mixed using a standard hand mixing procedure, in which the components are rapidly transferred between two syringes connected by 3-way stopcock. The physical measurements of the text preparations are set forth in Table 6. Specifically, the emulsions were mixed in various ways (see Table 6).

Oily phase vehicles Montanide ISA 25, 28 and 35 were admixed to the aqueous phase.

Montanide ISA 206, 206D and 264 were prepared by mixing after heating the aqueous phase and the oily vehicles to 30° C. in a water bath.

Aqueous phase was admixed to Montanide ISA 703, 719, and 720 to prepare injectable water-in-oil emulsions.

SBAS3 emulsion was prepared by diluting the stock aqueous phase in SB buffer, and admixing the aqueous phase to the SB62 adjuvant to produce an oil-in-water emulsion.

For further comparison, a water-in-oil emulsion was produced by adding half of the aqueous phase to Freund's adjuvant, mixing both portions and then adding the rest of the aqueous phase and mixing everything again.

One sample vial of each test emulsion was stored at 4° C. Five vials of each test emulsion were frozen either at about −70° C. (GMP Ultra-Low freezer) or at about −18° C. to −25° C. (standard, chest freezer). The actual temperature observed during the later storage was −23° C. (see Table 7).

When the vials were frozen thoroughly they were removed from the respective freezers and allowed to thaw to room temperature. One sample vial of each temperature and emulsion was retained for analysis and the remaining samples were refrozen at the respective aforementioned temperatures.

The test formulations were analyzed for appearance after storage at 4° C., as well as when first frozen, secondly after thawing but without shaking, and finally after shaking the vials with the thawed emulsions.

The results of the comparative study are displayed in Tables 7 and 8, below.

Summary of Comparative Test Emulsions

It is clear from the data that not all emulsion formulations show the stable storability according to this invention. Accordingly, the emulsions capable of withstanding freezing have been found to include Montanide ISA 25, 719 and 720 in addition to substances described in Examples 1 and 2.

Thus, an oil-in-water emulsion of anti-G17 immunogen in Montanide ISA 25 (emulsion #1) has been found stable at −70° C. and −23° C. Water-in-oil emulsions with Montanide ISA 703, 719 and 720 (emulsions 1, 8, 9 and 10, respectively) have been found stable during frozen storage at −70° C. and −23° C. However, of the other emulsions tested, none were stable at all three storage temperatures (i.e. −70° C., −23° C., and +4° C.)

TABLE 6
Physical Data of Emulsion with Different Vehicles

| | | | | | | | | | | Aqueous Phase | | | | | Emulsion | | | |
|---|---|---|---|---|---|---|---|---|---|---|---|---|---|---|---|---|---|---|
| | | | | | | | | | | | Target | | | | | | | |
| | | | | | | | | % Oil | % Aq | Aq | | Vol | Vol | | | | | Final |
| | | | | Specific | | | | Using Oil | using Oil's | phase | Dilution | Stock | Buffer | Total Vol | Aq phase | Vehicle | | Emulsion |
| | | Vehicle | | Gravity | Emulsion | Oil | Aq | Specific | Specific | conc | of stock | Needed | made | added | added | Total | conc | |
| Emulsion | Vehicle ID | Storage | Lot # | (g/ml) | Type | (%) | (%) | gravity | gravity | (mg/ml) | Aq | (ml) | (ml) | (ml) | (g) | (g) | Emulsion | (mg/ml) |
| 1 | Montanide ISA 25 | 2-8%° C. | 43222 | 0911 | O in W | 25 | 75 | 2679% | 7321% | 0683 | 293 | 2629 | 5071 | 7700 | 750 | 220 | 102 | 0500 |
| 2 | Montanide ISA 28D | 2-8%° C. | 03594 | 0932 | O in W | 25 | 75 | 2634% | 7366% | 0679 | 295 | 2613 | 5087 | 7700 | 750 | 250 | 102 | 0500 |
| 3 | Montanide ISA 35 | 2-8%° C. | 03694 | 0962 | O in W | 25 | 75 | 2573% | 7427% | 0673 | 297 | 2592 | 5100 | 7700 | 750 | 250 | 101 | 0500 |
| 4 | Montanide ISA 206 | 2-8%° C. | 43101 | 0850 | W-O-W | 50 | 50 | 5379% | 4621% | 1062 | 185 | 2013 | 2387 | 5200 | 500 | 500 | 108 | 0500 |
| 5 | Montanide 206D | 2-8%° C. | 03794 | 0869 | W-O-W | 50 | 50 | 5350% | 4650% | 1075 | 186 | 2796 | 2404 | 5200 | 500 | 500 | 108 | 0500 |
| 6 | Montanide 264 | 2-8%° C. | 03894 | 0921 | W-O-W | 20 | 50 | 5206% | 4794% | 1043 | 192 | 2712 | 2488 | 5200 | 500 | 500 | 104 | 0500 |
| 7 | Montanide 564 | 2-8%° C. | 03994 | 0916 | W in O | 50 | 50 | 5219% | 4781% | 1046 | 191 | 2719 | 2481 | 5200 | 500 | 500 | 105 | 0500 |
| 8 | Montanide ISA 703 | Rm | 71751 | 0853 | W in O | 70 | 30 | 7323% | 2677% | 1060 | 107 | 2988 | 0212 | 3200 | 300 | 700 | 112 | 0500 |
| | | temp | | | | | | | | | | | | | | | | |
| 9 | Montanide 719 | Rm | 74241 | 0085 | Win O | 60 | 40 | 6289% | 3711% | 1347 | 140 | 2388 | 1156 | 3544 | 334 | 502 | 90 | 0500 |
| | | temp | | | | | | | | | | | | | | | | |
| 10 | Montanide 720 | 2-8%° C. | 03494 | 0878 | Win O | 70 | 30 | 7266% | 2734% | 1829 | 109 | 2926 | 0274 | 3200 | 300 | 700 | 110 | 0500 |
| 11 | SBAS3 | 2-8%° C. | none listed | 1 | O in W | 50 | 50 | 5000% | 5000% | 1000 | 200 | 0850 | 0850 | 1700 | 15 | 150 | 30 | 0500 |
| 12 | Freunds Adjuvant | 2-8%° C. | 18K5674 | 0901 | Win O | 50 | 50 | 5260% | 4740% | 1055 | 190 | 2743 | 2457 | 5200 | 500 | 500 | 105 | 0500 |
| | (incomplete) | | | | | | | | | | | | | | | | | |
| 13 | Freunds Adjuvant | 2-8%° C. | 739732 | 0868 | Win O | 50 | 50 | 5353% | 46.47% | 1076 | 186 | 2798 | 2402 | 5200 | 500 | 500 | 108 | 0500 |
| | (complete) | | | | | | | | | | | | | | | | | |

Calculations:
% Oil using specific gravity = (% Oil/specific gravity)/((%Oil/specific gravity) + % Aqueous)
% Aqueous using Oil's specific gravity 100% = % Oil using specific gravity
Target Aqueous phase conc. (mg/ml) = target emulsion conc. (mg/ml)/% Aq. using oils specific gravity
Dilution of stock aq. = 2 mg/ml conc, stock/Target Aq. phase conc. (mg/ml)
Aqueous phase added (g) = % Aqueous X emulsion vol. (ml)
Vehicle added (g) % Oil X emulsion vol. (ml)
Final Aqueous conc. (mg/g) = original stock conc. X amount of stock)/(amount of stock + amount of PBS)
Emulsion vol. (ml) = Aqueous phase (g)/(1 g/ml)) + ((Vehicle (g)/specific gravity (g/ml)))
Emulsion conc. (mg/ml) = (Aqueous phase conc. (mg/ml) X Aqueous phase weight (g)/(1 g/ml) / Emulsion vol. (ml)
Aqueous phase conc. cheek (mg/ml) = ((Emulsion conc. (mg/ml) X ((Emulsion vol. (ml)))/(Aqueous phase weight (g)/(g/ml))

TABLE 7
Stability Test of Anti-G17 Emulsions with Different Vehicles Subjected to Freeze/Thaw Cycles
at −70° C. and −23° C.

| | | Emulsion | | Storage | Freeze/Thaw | |
|---|---|---|---|---|---|---|
| Emulsion | Vehicle I.D. | Type | Oil:Aq. | Temp. | 5 cycles | Stable at |
| 1 | Montanide ISA 25 | O in W | 25:75 | −70° C. | Stable | −70° C. |
| | | | | −23° C. | Stable | −23° C. |
| 2 | Montanide ISA 28D | O in W | 25:75 | −70° C. | Stable | −70° C. |
| | | | | −23° C. | Not Stable | |
| 3 | Montanide ISA 35 | O in W | 25:75 | −70° C. | Stable | −70° C. |
| | | | | −23° C. | Not Stable | |
| 4 | Montanide ISA 206 | W-O-W | 50:50 | −70° C. | Not Stable | |
| | | | | −23° C. | Not Stable | |
| 5 | Montanide ISA 206D | W-O-W | 50:50 | −70° C. | Not Stable | |
| | | | | −23° C. | Not Stable | |
| 6 | Montanide ISA 264 | W-O-W | 50:50 | −70° C. | Not Stable | |
| | | | | −23° C. | Not Stable | |
| 7 | Montanide ISA 564 | W in O | 50:50 | −70° C. | Not Stable | |
| | | | | −23° C. | Stable | −23° C. |
| 8 | Montanide ISA 703 | W in O | 70:30 | −70° C. | Stable | −70° C. |
| | | | | −23° C. | Stable | −23° C. |
| 9 | Montanide ISA 719 | W in O | 60:40 | −70° C. | Stable | −70° C. |
| | | | | −23° C. | Stable | −23° C. |
| 10 | Montanide ISA 720 | W in O | 70:30 | −70° C. | Stable | −70° C. |
| | | | | −23° C. | Stable | −23° C. |
| 11 | SBAS3 | W in O | 50:50 | −70° C. | Not Stable | |
| | | | | −23° C. | Stable | −23° C. |
| 12 | Freunds Adjuvant (Incomplete) | W in O | 50:50 | −70° C. | Not Stable | |
| | | | | −23° C. | Not Stable | |
| 13 | Freunds Adjuvant (Complete) | W in O | 50:50 | −70° C. | Stable | −70° C. |
| | | | | −23° C. | Stable | −23° C. |

TABLE 8
Stability Test of Anti-G17 Emulsions with Different Vehicles Stored at 4° C.

| | | Emulsion | | Storage | Day 0 (prior | | | |
|---|---|---|---|---|---|---|---|---|
| Emulsion | Vehicle I.D. | Type | Oil:Aq. | Temp. | to Storage) | Day 6 | Day 7 | Day 8 |
| 1 | Montanide ISA 25 | O in W | 25:75 | 4° C. | Stable | Stable | Stable | Stable |
| 2 | Montanide ISA 28D | O in W | 25:75 | 4° C. | Stable | Stable | Stable | Stable |
| 3 | Montanide ISA 35 | O in W | 25:75 | 4° C. | Stable | 5table | Stable | Stable |
| 4 | Montanide ISA 206 | W—O—W | 50:50 | 4° C. | Stable | Stable | Stable | Stable |
| 5 | Montanide ISA 206D | W—O—W | 50:50 | 4° C. | Stable | Stable | Stable | Stable |
| 6 | Montanide ISA 264 | W—O—W | 50:50 | 4° C. | Stable | Not Stable | Not Stable | Not Stable |
| 7 | Montanide ISA 564 | W in O | 50:50 | 4° C. | Stable | Not Stable | Not Stable | Not Stable |
| 8 | Montanide ISA 703 | W in O | 70:30 | 4° C. | Stable | Stable | Stable | Stable |
| 9 | Montanide ISA 719 | W in O | 60:40 | 4° C. | Stable | Not Stable | Not Stable | Not Stable |
| 10 | Montanide ISA 720 | W in O | 70:30 | 4° C. | Stable | Stable | Stable | Stable |
| 11 | SBAS3 | O in W | 50:50 | 4° C. | Stable | Stable | Stable | Stable |
| 12 | Freund's Adjuvant (Incomplete) | W in O | 50:50 | 4° C. | Not Stable | Not Stable | Not Stable | Not Stable |
| 13 | Freund's Adjuvant (Complete) | W in O | 50:50 | 4° C. | Stable | Not Stable | Not Stable | Not Stable |

What is claimed is:

1. A stable immunogenic emulsion composition suitable for frozen storage comprising: an emulsion which comprises an aqueous phase containing an immunomimic epitope conjugated to an immunogenic protein carrier; and a pharmaceutically acceptable oily vehicle, which supports a stable emulsion during frozen storage.

2. The composition according to claim 2, wherein the immunomimic epitope is a non-peptide moiety.

3. The composition according to claim 1, wherein the immunomimic epitope is a peptide.

4. The composition according to claim 3, wherein the immunomimic peptide contains the epitope of gastrin.

5. The composition according to claim 3 or 4, wherein the epitope is selected from the group consisting of gastrin 17 (“G17”) and gastrin 34 (“G34”).

6. The composition according to claim 1, wherein the immunomimic peptide contains the epitope of gonadotropin releasing hormone (GnRH).

7. The composition according to claim 1, wherein the immunogenic protein carrier is a foreign protein capable of evoking an effective immune response.

8. The composition according to claim 1 or 7, wherein the immunogenic protein is selected from the group consisting of diphtheria toxoid, tetanus toxoid, keyhole limpet hemocyanin, horseshoe crab hemocyanin, bovine serum albumin, extract of filamentous anycolate, extract of H. Pertussis, and dextran.

9. The composition according to claim 1, wherein the oily vehicle comprises squalene and/or squalane.

10. The composition according to claim 1 or 9, wherein the oily vehicle comprises Montanide ISA 703, Montanide ISA 25, Montanide ISA 719, or Montanide ISA 720.

11. The composition according to claim 1 or 9, wherein the oily vehicle comprises Montanide ISA 703.

12. The composition according to claim 1, wherein the emulsion comprises the conjugate as an aqueous solution and the oily vehicle in equal volumes.

13. The composition according to claim 1, wherein the emulsion comprises the conjugate as an aqueous solution and the oily vehicle in unequal volumes.

14. The composition according to claim 1, wherein the frozen storage lasts at least one year.

15. The composition according to claim 1, wherein the conjugate comprises a spacer peptide linking the immunomimic peptide to the immunogenic protein carrier.

16. An injectable immunogenic emulsion formulated for frozen storage comprising:
(a) an aqueous phase comprising a hormone peptide or fragment thereof which is conjugated to an immunogenic protein carrier; and
(b) an oily vehicle comprising a pharmaceutically acceptable oil or a mixture of pharmaceutically acceptable oils;
wherein the emulsion is stable during frozen storage.

17. The emulsion according to claim 16 wherein the aqueous phase and the oily vehicle are present at a weight ratio of about 70:30 for an oil-in-water emulsion.

18. The emulsion according to claim 16 wherein the aqueous phase and the oily vehicle are present at a weight ratio of about 30:70 for a water-in-oil emulsion.

19. The injectable emulsion of claim 16, wherein the frozen storage comprises a temperature ranging from about −18° C. to about −80° C.

20. The injectable emulsion of claim 16, wherein the frozen storage comprises a temperature of about −18° C.

21. The injectable of claim 16, wherein the frozen comprises a temperature of about -70° C.

22. The injectable emulsion of claim 16, wherein the long term frozen storage ranges from 3-12 months.

23. The injectable emulsion of claim 16, wherein after frozen storage at about -18° C, the emulsion exhibits a significant immunogenicity increase.

24. The injectable emulsion of claim 16, which has been sterile filtered.

25. The injectable emulsion of claim 16, wherein the hormone peptide or fragment thereof comprises an epitope of gastrin.

26. The injectable emulsion of claim 16, wherein the hormone peptide or fragment thereof comprises an epitope of G17.

27. The injectable emulsion of claim 16, wherein the hormone peptide or fragment thereof comprises an epitope of G34.

28. The injectable emulsion of claim 16, wherein the hormone peptide or fragment thereof comprises an epitope of the human GnRH.

29. The injectable emulsion of claim 16, wherein the immunogenic protein carrier is selected from diphtheria toxoid (“DT”), tetanus toxoid (“TT”), bovine serum albumin (“BSA”), keyhole limpet hemocyanin (“KHC”), extracts of H. Pertussis, extract of filamentous Amycolate, dextran, horseshoe crab hemocyanin, and ovalbumin.

30. The injectable emulsion of claim 16, wherein the emulsion comprises a G17 peptide fragment ranging from amino acid 1-9 of the amino terminal sequence which is conjugated at the ninth amino acid through a spacer to the DT carrier.

31. The injectable emulsion of claim 16, wherein after storage at about −18° C. the emulsion is effective in significantly increasing the anti-G17 antibody titer in an immunized animal.

32. The composition according to claim 1, wherein the emulsion stored at about −18° C. exhibits an increased immunogenicity.

33. A method for prolonged stable storage of an immunogen or vaccine comprising preparing a composition as claimed in claim 1.

34. The method for prolonged storage of an immunogen or vaccine according to claim 16, wherein the composition comprises an injectable formulated immunogenic emulsion which is stored frozen.
